SEQUENCE LISTING (14 sequences)

SEQ ID NO: 1 - protein, 43 aa, Homo sapiens
  MOD_RES (2)..(2): PHOSPHORYLATION
DSSEEKFLRRIGRFGYGYGPYQPVPEQPLYPQPYQPQYQQYTF

SEQ ID NO: 2 - protein, 42 aa, Homo sapiens
  MOD_RES (2)..(2): PHOSPHORYLATION
DSSEEKFLRRIGRFGYGYGPYQPVPEQPLYPQPYQPQYQQYT

SEQ ID NO: 3 - protein, 33 aa, Homo sapiens
  MOD_RES (2)..(2): PHOSPHORYLATION
DSSEEYGYGPYQPVPEQPLYPQPYQPQYQQYTF

SEQ ID NO: 4 - protein, 32 aa, Homo sapiens
  MOD_RES (2)..(2): PHOSPHORYLATION
DSSEEYGYGPYQPVPEQPLYPQPYQPQYQQYT

SEQ ID NO: 5 - protein, 21 aa, Artificial sequence
  Synthetic fragment of statherin
XXXXXXXXXXXXXXXYXYXXX

SEQ ID NO: 6 - protein, 21 aa, Artificial sequence
  Synthetic fragment of statherin
XXXXXXXXXXXXXXXYXYXXY

SEQ ID NO: 7 - protein, 21 aa, Artificial sequence
  Synthetic fragment of statherin
XXXXXXXXXXXGXFGYGYGPY

SEQ ID NO: 8 - protein, 21 aa, Artificial sequence
  Synthetic statherin peptide
DSSEEKFLRRIGRFGYGYGPY

SEQ ID NO: 9 - protein, 21 aa, Artificial sequence
  Synthetic statherin peptide
DDSEEKFLRRIGRFGYGYGPY

SEQ ID NO: 10 - protein, 21 aa, Artificial sequence
  Synthetic statherin peptide
DDDEEKFLRRIGRFGYGYGPY

SEQ ID NO: 11 - protein, 21 aa, Artificial sequence
  Synthetic statherin peptide
DDDDDKFLRRIGRFGYGYGPY

SEQ ID NO: 12 - protein, 21 aa, Artificial sequence
  Synthetic statherin peptide
DSEEEKFLRRIGRFGYGYGPY

SEQ ID NO: 13 - protein, 21 aa, Artificial sequence
  Synthetic statherin peptide
DEEEEKFLRRIGRFGYGYGPY

SEQ ID NO: 14 - protein, 21 aa, Artificial sequence
  Synthetic statherin peptide
EEEEEKFLRRIGRFGYGYGPY

(19) United States
(12) Patent Application Publication
(10) Pub. No.: US 2009/0074680 A1
(43) Pub. Date: Mar. 19, 2009

(54) STATHERIN PEPTIDE AND USE IN MEDICINE

(75) Inventors: Paul Anderson, London (GB); Jelena Kosoric, London (GB); Ralph Anthony David Williams, London (GB); Mark P. Hector, London (GB)

Correspondence Address: Pepper Hamilton LLP, 400 Berwyn Park, 899 Cassatt Road, Berwyn, PA, 19312-1183, US

(73) Assignee: QUEEN MARY & WESTFIELD COLLEGE, London (GB)

(21) Appl. No.: 11/909,342

(22) PCT Filed: Mar. 23, 2006
(86) PCT No.: PCT/GB2006/001085

§ 371 Date: Apr. 29, 2008

(30) Foreign Application Priority Data
Mar. 23, 2005 (GB) 0505975.3

(51) Int. Cl. A61K 38/17; A61P 1/14; C12P 21/02; C12N 15/11

(52) U.S. Cl. 424/54; 530/326; 514/13; 435/320.1; 435/69.1; 536/23.5

(57) ABSTRACT

A synthetic statherin peptide of general formula (I) X₁X₂X₃X₄X₅X₆X₇X₈X₉X₁₀X₁₁X₁₂X₁₃X₁₄X₁₅X₁₆X₁₇X₁₈X₁₉X₂₀X₂₁ as defined herein is provided. The peptide finds application in the treatment of dent disease, dry mouth syndrome and in the formulation of pharmaceutical preparation comprising the peptide.

The present invention relates to the provision of novel peptides based on native statherin protein and their use in medicine, with particular application in the preparation of artificial saliva.

Saliva is a glandular secretion that constantly bathes the teeth and the oral mucosa (Whelton, 1996). The protective role of saliva in enamel homeostasis is expressed as a synchronised action of its inorganic and organic content. High concentrations of calcium and phosphate in saliva are essential for protection against enamel dissolution. However, this level of supersaturation would suggest unwanted precipitation of calcium phosphates in salivary ducts, on enamel surfaces, and within the oral cavity. Salivary proteins prevent this unwanted effect by inhibition of primary and secondary precipitation of hydroxyapatite, and by binding calcium ions in solution.

Salivary protein statherin is produced by the acinar cells in the salivary glands (Hay and Bowen, 1996). Its molecular weight (Mᵣ) is 5380 and it contains 43 amino acids. It is a multifunctional molecule (Raj, 1992) involved in adsorption to hydroxyapatite (HAp), inhibition of primary and secondary precipitation of HAp and enamel (Schlesinger et al., 1987), able to help bacterial binding (Gibbons and Hay, 1988) and present in the pellicle very close to the enamel surface compared to other proteins identified (Schupbach et al., 2001).

Primary structures of statherin and its three variants (Jensen et al., 1991) are as follows:

DpSpSEEKFLRRIGRFGYGYGPYQPVPEQPLYPQPYQPQYQQYTF
(statherin)
DpSpSEEKFLRRIGRFGYGYGPYQPVPEQPLYPQPYQPQYQQYT_
(SY1)
DpSpSEE_{————————————}YGYGPYQPVPEQPLYPQPYQPQYQQYTF
(SV2)
DpSpSEE_{————————————}YGYGPYQPVPEQPLYPQPYQPQYQQYT_
(SV3)

This peptide exhibits charge polarity, which means that all charged amino acid residues except one, are located in the N-terminal region (Schlesinger and Hay, 1977; Lamkin and Oppenheim, 1993). As it is shown above, variants SV2 and SV3 are almost identical to statherin except for the 10 amino-acids fragment missing from lysine (K⁶) to tyrosine (Y¹⁶).

Inhibition of spontaneous precipitation involves interaction of the highly charged N-terminal of statherin with crystal nuclei (Hay et al., 1979), and the carboxy-terminal is oriented towards the bulk solution restricting the diffusion of ions towards the forming crystal nuclei (Schlesinger and Hay, 1977; Schwartz et al., 1992). In the case of inhibition of secondary precipitation, blocking of crystal growth sites (Brown 1966) is the most probable mechanism and only N-terminal of statherin is required (Hay and Moreno 1989, Raj et al., 1992).

Regarding calcium-binding, it is assumed that ˜10% of calcium (Ca²⁺) is directly bound by protein in stimulated saliva and ˜5% in unstimulated saliva (Hay, 1982). An additional mechanism of calcium-binding can be suggested knowing that salivary proteins tend to form salivary micelles, similar to milk proteins-caseins. Ca²⁺ would be loosely bound in protein-calcium-phosphate complexes which would act as a calcium phosphate reservoir (Reynolds, 1997). Similarity between those two groups, milk and salivary proteins, seems to be genetically related. Kawasaki and Weiss (2003) have found that genes for enamel matrix proteins (ameloblastin and enamelin), milk caseins and salivary proteins (statherin, histatin, acidic-proline rich proteins and mucin) belongs to the same gene family with common ancestor. Caseins and statherin have the same SXE motif (putative phosphorylation site-Fukae et al., 1996, Salih et al., 1998) or in other words Ser-Xaa-Glu (where Xaa represent any amino-acid) which have been found to be responsible for calcium-binding (Farrell and Thomson, 1988).

In addition, statherin, together with other salivary proteins, forms acquired enamel pellicle (Yao et al., 1992, Dawes 1963). The pellicle protects the dental enamel from dissolution and occlusal wear (Jensen, 1994).

Although much is understood about the chemical composition of saliva, there exists a need for artificial saliva compositions which can function effectively as the normal saliva produced by the body. In patients whose salivary glands have been removed surgically or which do not function appropriately, the only current treatment is to use artificial saliva solutions. Whilst such solutions are useful there are problems associated with their use.

Salivary glands may be removed in the course of surgery to treat cancer, or as the result of plastic surgery or other surgical interventions. Salivary glands may not function effectively in certain medical conditions, or for example following radiotherapy, where “dry mouth syndrome” is a recognised side effect.

Current treatments include saliva substitutes. Products available on the UK market include Glandosane® and Saliva Medac® among others available in France (Artisial®) and Australia (Oralube®). Kielbassa (2001) and colleagues tested the effect of saliva substitutes on mineral content of demineralised and sound enamel. Some of the products even increased mineral loss. Meyer-Lueckel et al. (2002) tested the same products and their influence on dentin. The conclusion was that saliva substitutes containing calcium, phosphate and fluoride would be strongly recommended in the cases of saliva reduction, but on the European market products containing those ions are not widely available. In addition, there are problems arising from the high concentrations of calcium and phosphate necessary to prevent enamel dissolution and enable remineralisation and while avoiding their unwanted precipitation.

Furthermore, the artificial saliva preparations are not able to prevent progressive demineralisation of the teeth. Demineralisation of the teeth has serious consequences for the subject concerned as the teeth are more exposed to developing dental diseases, such as dental caries.

It has been surprisingly found that a fragment of the statherin protein can act as a replacement for the natural form of the protein in an artificial saliva composition. The isolated synthetic peptide fragment retains its biological activity and is more stable than the native protein.

According to a first aspect of the invention, there is provided a peptide of general formula (I)

X₁X₂X₃X₄X₅X₆X₇X₈X₉X₁₀X₁₁X₁₂X₁₃X₁₄X₁₅X₁₆X₁₇X₁₈X₁₉X₂₀X₂₁ (I)

in which:
X₁, X₂, X₃, X₄ or X₅ each independently represents an acidic amino acid selected from the group consisting of serine, aspartic acid, or glutamic acid
X₆ represents a basic amino acid selected from the group consisting of lysine, arginine and histidine
X₇ or X₈ each independently represents a hydrophobic amino acid selected from the group consisting of glycine, alanine, phenylalanine, valine, leucine, isoleucine, tyrosine, tryptophan and methionine
X₉ or X₁₀ each independently represents a basic amino acid, preferably selected from the group consisting of lysine, arginine and histidine
X₁₁ or X₁₂ each independently represents a hydrophobic amino acid selected from the group consisting of glycine, alanine, phenylalanine, valine, leucine, isoleucine, tyrosine, tryptophan and methionine
X₁₃ represents a basic amino acid, preferably selected from the group consisting of lysine, arginine and histidine
X₁₄ or X₁₅ each independently represents a hydrophobic amino acid selected from the group consisting of glycine, alanine, phenylalanine, valine, leucine, isoleucine, tyrosine, tryptophan and methionine
X₁₆ represents tyrosine
X₁₇ represents a hydrophobic amino acid selected from the group consisting of glycine, alanine, phenylalanine, valine, leucine, isoleucine, tyrosine, tryptophan and methionine
X₁₈ represents tyrosine
X₁₉ or X₂₀ each independently represents a hydrophobic amino acid selected from the group consisting of glycine, alanine, phenylalanine, valine, leucine, isoleucine, tyrosine, tryptophan, proline and methionine
X₂₁ represents a hydrophobic amino acid selected from the group consisting of glycine, alanine, phenylalanine, valine, leucine, isoleucine, tyrosine, tryptophan and methionine.

In one embodiment, the peptide of formula (I) may be written as:

X₁X₂X₃X₄X₅(R/K)₆X₇X₈(R/K)₉(R/K)₁₀X₁₁X₁₂(R/K)₁₃X₁₄X₁₅(Y)₁₆X₁₇(Y)₁₈X₁₉X₂₀(Y)₂₁

where, “R/K” shows that either arginine or lysine may be present and “Y” shows that tyrosine is present. The numbering provided refers to the position number.

In one embodiment of the invention, peptide of general formula (I) may be composed of the following preferred residues:
X₇, X₈ and X₁₁ may each independently represent phenylalanine, leucine, or isoleucine,
X₁₂ may represent glycine,
X₁₄ may represent phenylalanine
X₁₅ and X₁₇ may each independently represent glycine,
X₁₉ may represent glycine,
X₂₀ may represent proline, and
X₂₁ may represent tyrosine.

Using the three letter and one letter codes the amino acids may also be referred to as follows: glycine (G or Gly), alanine (A or Ala), valine (V or Val), leucine (L or Leu), isoleucine (I or Ile), proline (P or Pro), phenylalanine (F or Phe), tyrosine (Y or Tyr), tryptophan (W or Trp), lysine (K or Lys), arginine (R or Arg), histidine (H or His), aspartic acid (D or Asp), glutamic acid (E or Glu), asparagine (N or Asn), glutamine (Q or Gln), cysteine (C or Cys), methionine (M or Met), serine (S or Ser) and Threonine (T or Thr). Where a residue may be aspartic acid or asparagine, the symbols Asx or B may be used. Where a residue may be glutamic acid or glutamine, the symbols Glx or Z may be used. References to aspartic acid include aspartate, and glutamic acid include glutamate, unless the context specifies otherwise.

Suitably, the hydrophobic amino acid may be selected from the group consisting of glycine (G), alanine (A), phenylalanine (F), valine (V), leucine (L) and isoleucine (I) tyrosine (Y), tryptophan (W) or proline (P).

The present invention also extends to variants of a peptide of general formula (I). An example of a variant of the present invention is a fusion protein comprising a peptide as defined above, apart from the substitution of one or more amino acids with one or more other amino acids. The skilled person is aware that various amino acids have similar properties. One or more such amino acids of a substance can often be substituted by one or more other such amino acids without eliminating a desired activity of that substance.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains).

Substitutions of this nature are often referred to as “conservative” or “semi-conservative” amino acid substitutions.

Amino acid deletions or insertions may also be made relative to the amino acid sequence for the fusion protein referred to above. Thus, for example, amino acids which do not have a substantial effect on the activity of the polypeptide, or at least which do not eliminate such activity, may be deleted. Such deletions can be advantageous since the overall length and the molecular weight of a polypeptide can be reduced whilst still retaining activity. This can enable the amount of polypeptide required for a particular purpose to be reduced—for example, dosage levels can be reduced.

Amino acid insertions relative to the sequence of the fusion protein above can also be made. This may be done to alter the properties of a substance of the present invention (e.g. to assist in identification, purification or expression, as explained above in relation to fusion proteins).

Amino acid changes relative to the sequence given above can be made using any suitable technique e.g. by using site-directed mutagenesis or solid state synthesis.

It should be appreciated that amino acid substitutions or insertions within the scope of the present invention can be made using naturally occurring or non-naturally occurring amino acids. Whether or not natural or synthetic amino acids are used, it is preferred that only L-amino acids are present.

Preferred peptides for use according to the present invention are as follows:

DSSEEKFLRRIGRFGYGYGPY
DDSEEKFLRRIGRFGYGYGPY
DDDEEKFLRRIGRFGYGYGPY
DDDDDKFLRRIGRFGYGYGPY
DSEEEKFLRRIGRFGYGYGPY
DEEEEKFLRRIGRFGYGYGPY
EEEEEKFLRRIGRFGYGYGPY

Optionally, the amino acid residues of the peptide may be modified, such as for example by phosphorylation, glycosylation etc. The phosphorylation status of an amino acid residue is shown as follows, where “pS” indicates a serine residue that is phosphorylated.

More preferred peptides of the invention are therefore:

DpSpSEEKFLRRIGRFGYGYGPY
DDpSEEKELRRIGRFGYGYGPY
DpSEEEKELRRIGRFGYGYGPY

The following peptide sequence may be referred to as peptide StN21 which is the N-terminal fragment of statherin which consists of 21 amino acids.

DpSpSEEKFLRRIGRFGYGYGPY

According to a second aspect of the invention, there is provided a nucleic acid sequence encoding a peptide of general formula (I).

The nucleic acid may be DNA, cDNA or RNA such as mRNA obtained by cloning or produced by chemical synthesis. The DNA may be single or double stranded. Single stranded DNA may be the coding sense strand, or it may be the non-coding or anti-sense strand.

According to a third aspect of the invention, there is provided a process for the preparation of a peptide of general formula (I), the process comprising ligating successive amino acid residues together. The process may be a solid-state synthesis process, for example “Fmoc” or “Bmoc” synthesis, (Fmoc Solid Phase Peptide Synthesis: A Practical Approach (Practical Approach S.), eds. W. Chan &, Peter White, Oxford University Press (2000)) or a liquid-phase chemical synthesis reaction (Combinatorial Chemistry: A Practical Approach, ed. Hicham Fenniri, Oxford University Press (2000)) using standard procedures. In some instances, fragments may be synthesised using solid-state methods and then coupled together in solution. Peptides can be synthesized from the carbonyl group side to amino group side of the amino acid chain in this method, although peptides are synthesized in the opposite direction in cells. In such methods, an amino-protected amino acid is bound to a substrate bead (i.e. a resin bead), forming a covalent bond between the carbonyl group and the resin. The amino group is then de-protected and reacted with the carbonyl group of the next amino-protected amino acid. The cycle is repeated as often as required in order to form the desired peptide chain. The synthesized peptide is then cleaved from the bead at the end of the procedure. The protecting groups for the amino groups mostly used in this peptide synthesis are 9-fluorenylmethyloxycarbonyl group (“Fmoc”) and t-butyloxycarbonyl (“Boc”). The Fmoc group is removed from the amino terminus with base while the Boc group is removed with acid.

Alternatively, the process may comprise the expression of a nucleic acid construct according to the second aspect of the invention in a suitable host cell. Expression of the nucleic acid construction may suitably be achieved by transfecting the host cell with a vector comprising a nucleic acid of the second aspect of the invention.

The vector may also comprise suitable regulatory and/or promoter sequences to assist in expression of the sequence.

A vector as described above may be, for example, an expression vector, and may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host, may be used for expression in this regard.

Such vectors or nucleic acid constructs may suitably include a promoter or other regulatory sequence which controls expression of the nucleic acid. Promoters and other regulatory sequences which control expression of a nucleic acid have been identified and are known in the art. The person skilled in the art will note that it may not be necessary to utilise the whole promoter or other regulatory sequence. Only the minimum essential regulatory element may be required and, in fact, such elements can be used to construct chimeric sequences or other promoters. The essential requirement is, of course, to retain the tissue and/or temporal specificity. The promoter may be any suitable known promoter, for example, the human cytomegalovirus (CMV) promoter, the CMV immediate early promoter, the HSV thymidinekinase, the early and late SV40 promoters or the promoters of retroviral LTRs, such as those of the Rous Sarcoma virus (RSV) and metallothionine promoters such as the mouse metallothionine-I promoter. The promoter may comprise the minimum comprised for promoter activity (such as a TATA elements without enhancer elements) for example, the minimum sequence of the CMV promoter. Preferably, the promoter is contiguous to the nucleic acid sequence.

As stated herein, the nucleic acid construct of the invention may be in the form of a vector. Vectors frequently include one or more expression markers which enable selection of cells transfected (or transformed) with them, and preferably, to enable a selection of cells containing vectors incorporating heterologous DNA. A suitable start and stop signal will generally be present.

One embodiment of the invention relates to a cell comprising the nucleic acid construct of the second aspect of the invention. The cell may be termed a “host” cell, which is useful for the manipulation of the nucleic acid, including cloning. Alternatively, the cell may be a cell in which to obtain expression of the nucleic acid. Representative examples of appropriate host cells for expression of the nucleic acid construct of the invention include virus packaging cells which allow encapsulation of the nucleic acid into a viral vector; bacterial cells, such as Streptococci, Staphylococci, E. coli, Streptomyces and Bacillus Subtilis; single cells, such as yeast cells, for example, Saccharomyces Cerevisiae, and Aspergillus cells; insect cells such as Drosophila S2 and Spodoptera Sf9 cells, animal cells such as CHO, COS, C127, 3T3, PHK.293, and Bowes Melanoma cells and other suitable human cells; and plant cells e.g. Arabidopsis thaliana.

Induction of an expression vector into the host cell can be affected by calcium phosphate transfection, DEAE-dextran mediated transfection, microinjection, cationic—lipid-mediated transfection, electroporation, transduction, scrape loading, ballistic introduction, infection or other methods. Such methods are described in many standard laboratory manuals, such as Sambrook et al, Molecular Cloning, a Laboratory Manual, Second Edition, Coldspring Harbor Laboratory Press, Coldspring Harbor, N.Y. (1989).

Mature proteins can be expressed in host cells, including mammalian cells such as CHO cells, yeast, bacteria, or other cells under the control of appropriate promoters. Cell-free translation systems can be employed to produce such proteins using RNAs derived from the nucleic acid construct of the third aspect of the present invention. Appropriate cloning and expression vectors for use with prokaryotic and eukaryotic hosts are described by Sambrook et al, Molecular Cloning, a Laboratory Manual, Second Edition, Coldspring Harbor Laboratory Press, Coldspring Harbor, N.Y. (1989).

Proteins can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxyapatite chromatography, high performance liquid chromatography and lectin chromatography. For therapy, the nucleic acid construct e.g. in the form of a recombinant vector, may be purified by techniques known in the art, such as by means of column chromatography as described in Sambrook et al, Molecular Cloning, a Laboratory Manual, Second Edition, Coldspring Harbor Laboratory Press, Coldspring Harbor, N.Y. (1989).

Optionally, the resultant peptide synthesized by chemical means or expressed as described above may be phosphorylated at one or more hydroxyl groups in the molecule, preferably on the serine residues at positions 2 and 3. The phosphorylation may be carried out as part of the expression of the peptide, or subsequently in vitro.

According to a fourth aspect of the invention, there is provided a peptide of the first aspect for use in the prevention and/or treatment of demineralisation of teeth. Demineralisation of teeth may be defined as a loss of hydroxyapatite from the teeth.

This aspect of the invention therefore also extends to a method of treatment of or prevention of demineralisation of teeth in a subject, comprising administration to the subject of a peptide of general formula (I). In an alternative embodiment, the invention may be seen as providing the use of a peptide of general formula (I) in the preparation of a medicament for the treatment and/or prevention of demineralisation of teeth.

According to a fifth aspect of the present invention, there is provided a peptide of general formula (I) for use in the treatment and/or prevention of a dental disease. The dental disease may be dental caries or dental erosion.

This aspect of the invention therefore also extends to a method of treatment of or prevention of a dental disease in a subject, comprising administration to the subject of a peptide of general formula (I). In an alternative embodiment, the invention may be seen as providing the use of a peptide of general formula (I) in the preparation of a medicament for the treatment and/or prevention of a dental disease.

According to a sixth aspect of the present invention, there is provided a peptide of general formula (I) for use in the treatment and/or prevention of dry mouth syndrome.

There are a variety of a reasons for dry mouth syndrome, such as a side effect of a medication (i.e. use of diuretics or antidepressant therapy), salivary gland's loss of salivary gland function or dysfunction (as a consequence of diabetes, Sjögren syndrome—an autoimmune disease) or radiotherapy as a treatment for malignant tumors of the head and neck (Sreebny, 1996). Also it may occur as a result of loss of gland tissue related to aging process. As a consequence of reduction (or complete loss) of saliva, oral tissue are considerably more susceptible to infections and diseases such as dental caries, tooth erosion, mucositis and mucosal infections, including candidiasis; and speech, eating and swallowing become difficult and painful (Bennick and Hand, 2003), resulting in significant reduction in quality of life.

This aspect of the invention therefore also extends to a method of treatment of or prevention of dry mouth syndrome in a subject, comprising administration to the subject of a peptide of general formula (I). Since dry mouth syndrome is commonly associated with radiotherapy, an alternative embodiment of this aspect of the invention may extend to the use of such a peptide in a method of radiotherapy of a subject, for example radiotherapy of the head and neck of the subject.

In an alternative embodiment, the invention may be seen as providing the use of a peptide of general formula (I) in the preparation of a medicament for the treatment and/or prevention of dry mouth syndrome.

The peptides of the present invention may be employed alone or in conjunction with other compounds, such as therapeutic compounds, e.g. anti-inflammatory drugs, cytotoxic agents, cytostatic agents or antibiotics. The nucleic acid constructs and proteins useful in the present invention are preferably provided in an isolated form, and preferably are purified to homogeneity.

As used herein, the term “treatment” includes any regime that can benefit a human or a non-human animal. The treatment of “non-human animals” extends to the treatment of domestic animals, including horses and companion animals (e.g. cats and dogs) and farm/agricultural animals including members of the ovine, caprine, porcine, bovine and equine families. The treatment may be in respect of any existing condition or disorder, or may be prophylactic (preventive treatment). The treatment may be of an inherited or an acquired disease. The treatment may be of an acute or chronic condition. Preferably, the treatment is of a condition/disorder associated with inflammation.

The present invention may also find application in veterinary medicine for treatment/prophylaxis of domestic animals including horses and companion animals (e.g. cats and dogs) and farm animals which may include members of the ovine, porcine, caprine, bovine and equine families.

According to a seventh aspect of the invention, there is provided a pharmaceutical composition comprising a peptide of general formula (I).

Suitably, the pharmaceutical composition may be formulated for oral administration or for topical administration in the mouth cavity or on the teeth or gums by way of a coating. The pharmaceutical composition may be an artificial saliva, a mouth wash (buccal wash), tooth paste or cream, moisturiser, chewing gum, drink or other oral healthcare preparation.

An artificial saliva according to this aspect of the invention may comprise a peptide of general formula (I), an inorganic acid Group I or Group II metal ion salt, and water optionally also including flavouring, and/or preservative.

Suitably, the inorganic acid Group I or Group II metal ion salt may comprise one or more of potassium chloride (KCl), sodium chloride (NaCl), magnesium chloride (MgCl₂), calcium chloride (CaCl₂), potassium hydrogen phosphate (K₂HPO₄), potassium dihydrogen phosphate (KH₂PO₄), potassium thiocyanate (KSCN).

Suitably, the composition can be formulated using magnesium chloride as magnesium chloride hexahydrate (MgCl₂.6H₂O) and the calcium chloride as calcium chloride dihydrate (CaCl₂.2H₂O)

The pH of the composition when formulated using water may be suitably in the range pH 7.5 to pH 7.0, optionally suitably buffered using available physiological buffer solutions. Normal oral pH is 7.2 and the composition may be formulated at such a pH.

The peptides of the present invention are stable at acidic pH values. When food or drink is taken by a subject, the pH in the mouth can be lowered. However, the peptides of the present invention are stable at lower pH values down to pH 5.5 or lower, for example pH 5.5 to pH 4.5, or even as low as pH 4.3.

In addition, during a carious attack, where the acids are mainly lactic acid (pK 3.9) and acetic acid (pK 4.8), the pH is thought to drop to around pH 4.5. The buffering capacity of natural saliva tends to prevent further falls in pH, but in an artificial saliva the use of suitable buffering salts is preferred, as described above.

At present, no proteins involved in biomineralisation are present in artificial saliva due to concerns over biological activity and stability. The present invention overcomes such issues by providing a biologically active statherin derivative that is more stable than the native form of the protein.

Pharmaceutical compositions in accordance with this aspect of the invention may comprise other pharmaceutically active substances, such as anti-bacterial, anti-viral, anti-fungal, analgesic substances. The composition may also comprise pharmacologically acceptable salts such as fluoride salts or phosphate salts, for example a fluoride salt or a phosphate with an alkali metal or an alkaline earth metal, e.g. sodium fluoride (NaF). The pharmaceutical composition may be formulated using any convenient adjuvant and/or physiologically acceptable diluents. Other components may also be present in order to improve “mouthfeel” of the composition, such as sorbitol, xanthan gum, guar gum, and/or cellulose derivatives such as hydroxypropylmethylcellulose (HPMC), sodium carboxymethyl cellulose etc.

Mouthwashes may be formulated as desired using buffered physiologically acceptable media such as water, saline solution, alcohol (ethanol) and components such as sweeteners including saccharine, xylitol, preservatives including benzalkonium chloride, sodium benzoate, flavourings including menthol, enzymes to help prevent bacterial growth, such as lysozyme, lactoperoxidase and/or glucose oxidase, detergents, anti-bacterial peptides such as lactoferrin, as well as other components to improve “mouthfeel” as above.

In a preferred embodiment of the invention, there is provided an artificial saliva having the following composition

### Artificial Saliva-1

A suitable artificial saliva may be composed of:
Peptide of general formula (I), Potassium chloride (KCl), Sodium chloride (NaCl), Magnesium chloride (MgCl₂), Calcium chloride (CaCl₂), Potassium hydrogen phosphate (K₂HPO₄), Potassium dihydrogen phosphate (KH₂PO₄), Methyl p-hydroxybenzoate, Flavouring, 70% sorbitol, Sodium carboxymethyl cellulose, Sodium fluoride, (NaF), Water.

Suitable concentration ranges may be:

| | | |
|---|---|---|
| Peptide of general formula (I) | 50.0 to 200.0 | mg/l |
| Potassium chloride (KCl) | 0.5 to 2.0 | g/l |
| Sodium chloride (NaCl) | 0.5 to 1.5 | g/l |
| Magnesium chloride (MgCl₂) | 0.05 to 0.20 | g/l |
| Calcium chloride (CaCl₂) | 0.05 to 0.50 | g/l |
| Potassium hydrogen phosphate (K₂HPO₄) | 0.5 to 1.0 | g/l |
| Potassium dihydrogen phosphate (KH₂PO₄) | 0.25 to 1.0 | g/l |
| Methyl p-hydroxybenzoate | 1.25 to 2.50 | g/l |
| Flavouring | 2.5 to 5.0 | g/l |
| 70% sorbitol | 35.0 to 50.0 | g/l |
| Sodium carboxymethyl cellulose | 7.0 to 15.0 | g/l |
| Sodium fluoride (NaF) | 2.5 to 5.0 | mg/l |

### Artificial Saliva-2

A suitable artificial saliva may be composed of:
Peptide of general formula (I), Carboxymethylcellulose, Sorbitol, Potassium chloride (KCl), Sodium chloride (NaCl), Magnesium chloride hexahydrate (MgCl₂.6H₂O), Calcium chloride dihydrate (CaCl₂.2H₂O), Potassium hydrogen phosphate (K₂HPO₄), Potassium thiocyanate (KSCN), Water.

Suitable concentration ranges may be:

| | | |
|---|---|---|
| Peptide of general formula (I) | 50.0 to 200.0 | mg/l |
| Carboxymethylcellulose | 5 to 15 | g/l |
| Sorbitol | 1 to 10 | g/l |
| Potassium chloride (KCl) | 1 to 5 | g/l |
| Sodium chloride (NaCl) | 0.5 to 1.5 | g/l |
| Magnesium chloride hexahydrate (MgCl₂•6H₂O) | 0.01 to 0.10 | g/l |
| Calcium chloride dihydrate (CaCl₂•2H₂O) | 0.1 to 0.5 | g/l |
| Potassium hydrogen phosphate (K₂HPO₄), | 0.1 to 0.5 | g/l |
| Potassium thiocyanate (KSCN) | 0.05 to 0.20 | g/l |

In an alternative embodiment of the invention, the peptide may be prepared as a toothpaste, as follows:
Peptide of general formula (I), antibacterial enzyme or peptide (e.g. Lactoperoxidase, Glucose oxidase, Lactoferrin, Lysozyme), Sodium monofluorophosphate, Sorbitol, Glycerin, Calcium pyrophosphate, Hydrated silica, Isoceteth-20, Cellulose gum, Flavour, Sodium benzoate, β-D-Glucose, Potassium thiocyanate, Calcium lactate.

A mouthwash of the invention may be prepared as follows:
Peptide of general formula (I), anti-bacterial enzyme or peptide (e.g. Lysozyme, Lactoferrin, Glucose oxidase, Lactoperoxidase), Water, Hydrogenated starch, Propylene glycol, Hydroxyethyl cellulose, Aloe vera, Flavour, Poloxamer 407, Calcium lactate, Zinc gluconate, Sodium benzoate, Benzoic acid, Potassium thiocyanate.

A topical cream or moisturiser for use in the oral cavity may be prepared as follows:
Peptide of general formula (I), anti-bacterial enzyme or peptide (e.g. Lactoperoxidase, Lysozyme, Glucose oxidase, Lactoferrin), Hydrogenated starch, Xylitol, Hydroxyethyl cellulose, Glyceryl polymethacrylate, Aloe Vera, Potassium thiocyanate, β-D-Glucose.

Preferred features for the second and subsequent aspects of the invention are as for the first aspect mutatis mutandis.

The present invention will now be described by way of reference to the following Examples which are present for the purposes of reference only and are not to be construed as being limiting on the invention. In the Examples, reference is made to a number of drawings in which:

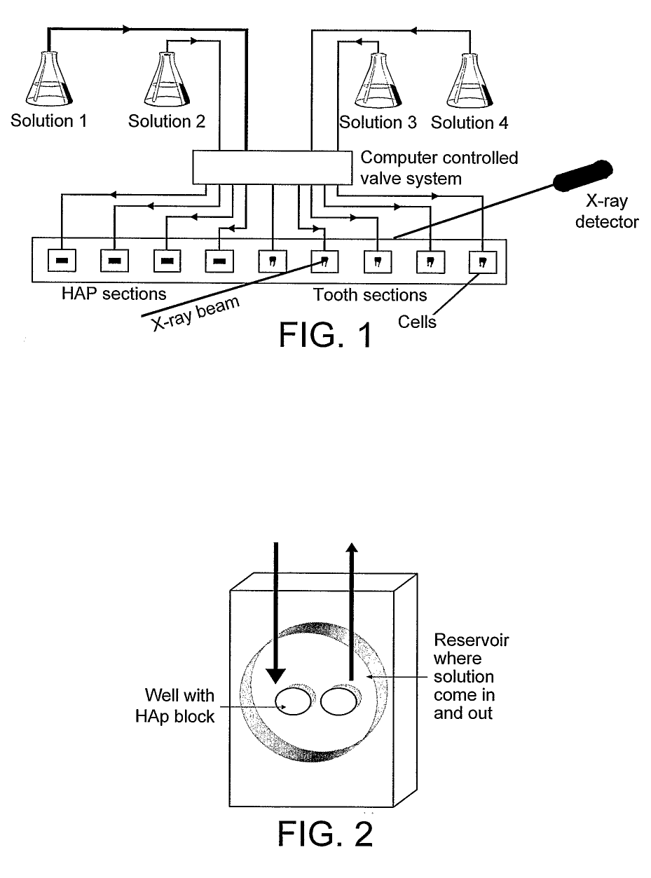

FIG. 1 shows a schematic scanner stage with SMR cells and circulating solutions.

FIG. 2 shows a schematic drawing of SMR cell

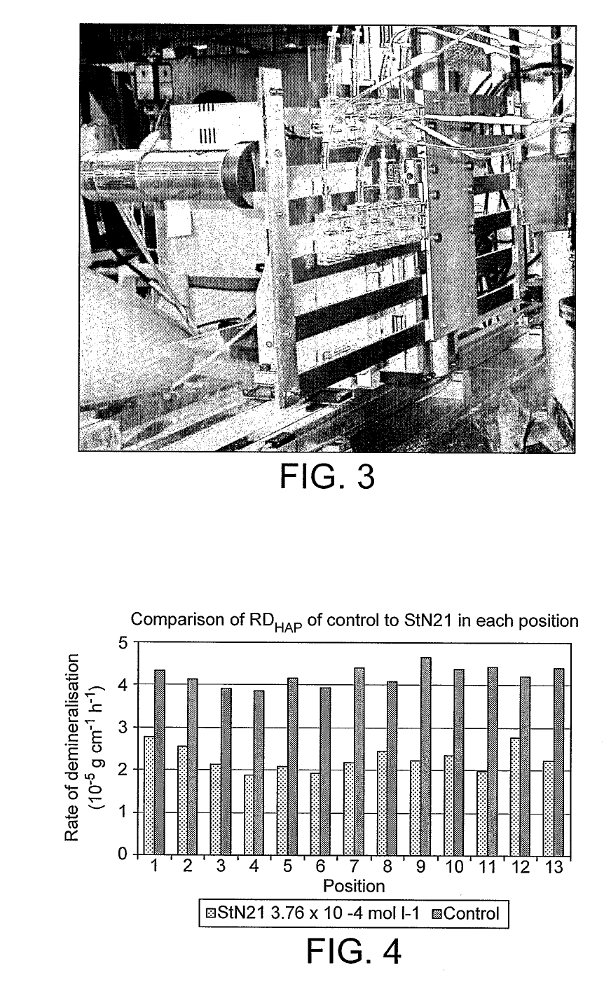

FIG. 3 shows a photograph of SMR: X-ray source, detector, SMR stage with SMR cells

FIG. 4 shows the rate of demineralisation of HAp (RD_{HAp}) at each position for two samples, one being a control and the other coated with StN21 peptide. Each bar represents RD_{HAp} at a single position of scanning.

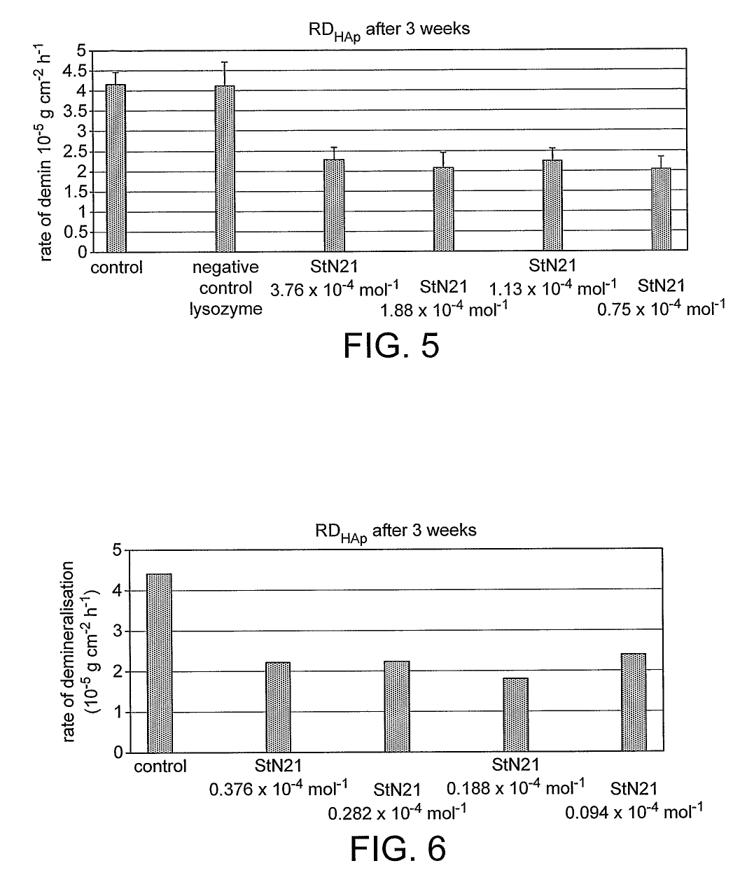

FIG. 5 shows the average rate of demineralisation of HAp coated with StN21 in the range 3.76 to 0.75×10⁻⁴ mol l⁻¹ compared with uncoated control and lysozyme (negative control).

FIG. 6 shows the average rate of demineralisation of HAp coated with StN21 in the range 0.376 to 0.094×10⁻⁴ mol l⁻¹ compared with control, using lower concentrations of StN21.

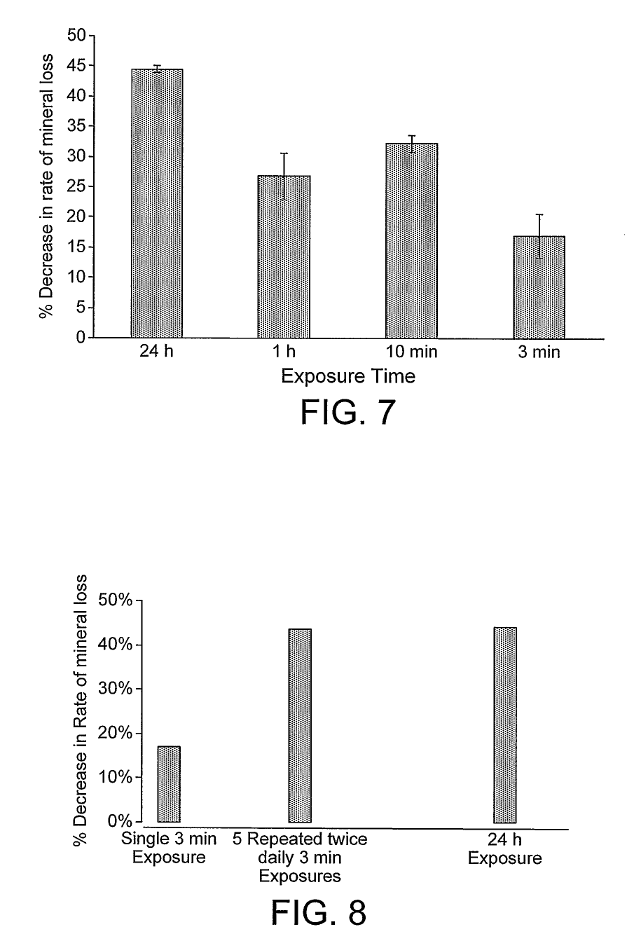

FIG. 7 shows the percentage (%) decrease in rate of demineralisation of Hydroxyapatite (HAp) with time of exposure of STN21 (at a concentration of 1.88×10⁻⁴ mol l⁻¹)

FIG. 8 shows a comparison of percentage (%) decrease in rate of demineralisation of Hydroxyapatite (HAp) after 3 min exposure of STN21 (at a concentration of 1.88×10⁻⁴ mol l⁻¹) repeated twice daily as “refreshment treatments”.

### EXAMPLE 1

### Studies on Demineralisation

HAp pellets are used in many studies instead of enamel blocks to prevent effect of enamel organic impurities and its natural surface on rate of demineralisation (Anderson et al., 2004). Statherin could not be isolated in a sufficiently pure form from saliva. Therefore N-terminal 21 residue fragment of statherin (StN21) was chemically synthesised using standard FastMoc synthesis (Fields et al., 1990). Also SXE motif, responsible for calcium binding in caseins, is present in this 21 amino-acid fragment of statherin. To exclude the possibility that StN21 act solely as a semi-permeable membrane, lysozyme was used as a negative control. It was found to bind to HAp surface but it does not influence rate of demineralisation of HAp (Poumier et al., 1996)

The aim of this study was to observe the influence of a StN21 peptide coating on the rate of demineralisation of HAp.

The 21 amino-acid peptide identical to N-terminus of statherin was purchased from Peptide Protein Research Ltd (UK). It was chemically synthesised using standard solid phase synthetic techniques using FastMoc chemistry on an automated peptide synthesiser with purity >95%. Compound identity was confirmed by Mass spectrometry.

Hydroxyapatite (HAp) pellets of 20 wt % porosity (Plasma Biotal Ltd, UK) were sliced into 3×3 mm blocks with 2 mm thickness using Microslice 2 cutter (Malvern Instruments Ltd.). Entire surface of the block, except one side was coated with acid resistant nail varnish. Ten HAp blocks were put into a solution saturated with respect to HAp for 24 hours to equilibrate. After equilibration, four HAp blocks were exposed for 24 h to 0.5 ml of four different concentrations of StN21 peptide dissolved in modified buffer (100 mmol⁻¹ NaCl, 40 mmol⁻¹ KCl, 4.3 mmol⁻¹ Na₂HPO₄, and 1.4 mmol⁻¹ KH₂PO₄, pH 7.4). The concentrations of StN21 peptide were 0.75, 1.13, 1.88 and 3.76×10⁻⁴ mol l⁻¹. As a negative control one HAp pellet was put into 0.5 ml of solution containing lysozyme at concentration of 0.11×10⁻⁴ mol l⁻¹.

Scanning microradiography (SMR) is a technique for observing very subtle changes in mineral mass during re and demineralisation of enamel and HAp. It allows observation period of up to 1000 h without disruptions to the specimen. Also, conditions can be modified and the effect of applied modifications on the samples can be monitored. SMR (Anderson et al., 2004) is an X-ray photon counting technique where the intensity of 15 μm X ray beam is attenuated by passing through the specimen and the transmitted photons are counted by an X-ray detector. FIG. 1 shows a schematic scanner stage with SMR cells and circulating solutions and FIG. 3 a photograph of SMR.

HAp blocks were placed in the wells of SMR cells (FIG. 2). Each SMR cell contained one HAp block coated with protein and one as a control. As an additional control, in one SMR cell two uncoated HAp blocks were placed in one SMR cell. The reservoir of the SMR cell is approximately 1.2 ml and solution is circulating past the HAp blocks at 0.4 ml min⁻¹.

Demineralising solution was 0.1 mol l⁻¹ acetic acid, buffered to pH 4.5 with 1 mol l⁻¹ NaOH; 1 mmol l⁻¹ calcium and 0.6 mmol l⁻¹ phosphate ions.

SMR was used to monitor changes in mineral mass at 12 positions 10 μm apart, along 2 lines in each well. Scanning time was 30 s per position. Total period of observation was 3 weeks. The rate of mineral change at each position was calculated by linear least squares fitting.

### Results

FIG. 4 shows the rate of demineralisation of HAp (RD_{HAp}) at each position for two samples, one being a control and the other coated with StN21 peptide. Each bar represents RD_{HAp} at a single position of scanning.

RD_{HAp} of coated HAp is significantly lower than the control at each position of scanning. The average rate of mineral change was calculated for each sample and is presented in FIGS. 5 and 6.

There is no difference in the RD_{HAp} of the control and the negative control. RD_{HAp} of HAp coated with StN21 peptide is significantly lower compared to control. However, no significant difference can be observed in the RD_{HAp} among the samples coated with different concentration of StN21 although a trend of decrease of RD_{HAp} with increasing StN21 concentration exist (2.2, 1.9, 1.7 and 1.8×10⁻⁵ g cm⁻² h⁻¹ at StN21 concentrations 0.75, 1.13, 1.88 and 3.76×10⁻⁴ mol l⁻¹, respectively).

FIGS. 5 and 6 show a significant decrease in the RD_{HAp} of HAp blocks coated with StN21 compared to control. This effect of StN21 peptide on RD_{HAp} might be explained either by the presence of bound protein on the surface of HAp or its influence on the driving force of dissolution. The decrease of the RD_{HAp} is not simply due to bound protein on the HAp surface; FIG. 5 shows that lysozyme coated HAp blocks (negative control) produced very similar results to uncoated (control) blocks. Lysozyme is known to bind to HAp surface (Poumier et al., 1996) but it does not influence the RD_{HAp}. When bound to an enamel surface, statherin may undergo conformational changes due to the change in pH and release bound Ca²⁺, similar to enamel matrix proteins when regulating calcium concentration in the extracellular enamel fluid (Yamakoshi et al., 2001) Therefore, the effect of statherin can be twofold: by increasing Ca²⁺ concentration close to enamel surface and as a semi-permeable membrane which prevents diffusion of Ca²⁺ ions released by demineralisation into a bulk solution. This will make the driving force for dissolution to lose its potential and consequently will decrease the RD_{HAp}. This mechanism would keep saliva surrounding enamel supersaturated and prevent dissolution of enamel (Reynolds, 1997).

However, the concentrations of StN21 showed the same effect. The concentrations of StN21 shown in FIG. 5 are higher than the average concentration of statherin found in saliva (0.367×10⁻⁴ mol l⁻¹), whereas in FIG. 6, the concentrations are equal or lower than found in saliva. The next step will be to prepare lower concentrations of StN21 and find the lowest concentration which will still show the same effect on the RD_{HAp}.

At 9.4×10⁻⁶ molar, equivalent to 25 mg/litre, the StN21 peptide was effective.

### EXAMPLE 2

### Artificial Saliva Preparations

Artificial saliva preparations which contain a peptide of the present invention may be made as follows. Peptide StN21 is the N-terminal fragment of statherin which consists of 21 amino acids.

### Artificial Saliva Preparation-1

| | | |
|---|---|---|
| Peptide StN21 | 400.0 | mg |
| KCl | 2.498 | g |
| NaCl | 3.462 | g |
| MgCl₂ | 0.235 | g |
| CaCl₂ | 0.665 | g |
| K₂HPO₄ | 3.213 | g |
| KH₂PO₄ | 1.304 | g |
| Methyl p-hydroxybenzoate | 8 | g |
| Flavouring | 16 | g |
| 70% sorbitol | 171 | g |
| Na-carboxymethyl cellulose | 40 | g |
| NaF | 17.68 | mg |
| Water | 4 | L |

### Artificial Saliva Preparation-2

| | | |
|---|---|---|
| Peptide StN21 | 100.0 | mg |
| Carboxymethylcellulose | 10 | g |
| Sorbitol | 3 | g |
| KCl | 1.2 | g |
| NaCl | 0.843 | g |
| MgCl₂•6H₂O | 0.051 | g |
| CaCl₂•2H₂O | 0.146 | g |
| K₂HPO₄ | 0.342 | g |
| KSCN | 0.1 | g |
| Water | 1 | L |

### EXAMPLE 3

### Activity of StN21 Peptide

The activity of StN21 was tested as follows in an experimental system.

STN21 was synthesised using Fmoc solid state synthesis. Hydroxyapatite (HAp) pellets were cut into 5×5×2 mm blocks and covered with acid-resistant varnish leaving one surface exposed and immersed in STN21 solution (1.88×10⁻⁴ mol l⁻¹) for 3 min. The pellets were then located in scanning microradiography (SMR) cells. Demineralising solution (0.1 mol l⁻¹ acetic acid, pH 4.5) was circulated (0.4 ml min⁻¹) for 3 weeks.

Five samples were exposed to further twice daily 3 min. immersions in STN21 solution. Changes in mineral mass were measured using SMR for 28 days.

HAp pellets coated with STN21 for one 3 min period only showed a 17% decrease in rate of mineral loss compared to a non-coated control. Samples exposed to repeated applications of StN21 peptide showed a 45% decrease in demineralisation rate.

Repeated applications of StN21 resulted in a significantly reduced rate of mineral loss in HAp samples maintained under demineralising conditions. This in vitro data strongly suggests that there is value in investigating the use of StN21 in vivo for the prevention and early treatment of dental caries and enamel erosion as part of further development of the minimally invasive approach to the management of enamel caries and erosion.

The results are shown in FIGS. 7 and 8. FIG. 7 shows the effects after different time intervals and FIG. 8 shows the effect of 5 repeated twice daily 3 min exposures (with 24 h for comparison).

Repeated application of the peptide is therefore as effective as a continuous application over 24 hours.

### REFERENCES

Anderson et al Arch Oral Biol 49: 199-207 (2004)
Anderson et al Imaging & Microscopy 02: 22-24 (2003)
Bennick, A. and Hand, A. R., in Ten Cate's Oral Histology: Development, Structure and Function, ed. Antonio Nanci, Mosby, Inc (2003)
Brown, W. E. & Wallace, B. M. Ann. N.Y. Acad. Sci., 131; 2: 690-693 (1965)
Dawes et al British Dent J, 115, 65-68 (1963)
Farrell, H. M. and Thompson, M. P. in Calcium-Binding proteins, ed. Thompson M P (CRC Boca Raton, Fla.) vol II, pp 117-37 (1988)
Fields et al Peptide Research 4, 95-101 (1990)
Fukae et al Adv Dent Res 10: 111-118 (1996)
Gibbons R. J., Hay D. I. Infect Immunol 56: 439-45 (1988)
Greenby T. H. Eur J Oral Sci 104: 221-228 (1996)
Hay et al Inorg Perspectives Biol Med 2, 271-285 (1979)
Hay et al Calcif Tissue Int, 34; 6: 531-538 (1982).
Hay, D. I. and Moreno, E. C. in “Human Saliva: Clinical Chemistry and Microbiology”, 2^{nd} ed., 55-91 Ed Tenovuo J O, CRC Press INC., Boca Raton, Fla. (1989)
Hay, D. I., Bowen, W. in “The functions of salivary proteins”. Edgar, W. M., O'Mullane, D. M., Saliva and oral health. Br Dent J 2^{nd} ed. 105-122 (1996).
Jensen J L et al Arch Oral Biol 36: 529-534 (1991).
Jensen, J. L. in “Human saliva: Biochemical and physiological aspects of some components” Faculty of Dentistry, University of Oslo, Norway (1994)
Kawasaki, K. and Weiss, K. M. Proceedings of the National Academy of Sciences of the United States of America 100; 7: 4060-65 (2003)
Kielbassa et al Support Care Cancer 9: 40-47 (2001)
Lamkin, M. S., Oppenheim, F. G., Crit. Rev Oral Biol Med 4: 251-259 (1993)
Meyer-Lueckel et al Oral Diseases 8: 192-98 (2002)
Poumier et al Colloids Surfaces B: Interfaces 7: 1-8 (1996)
Raj et al J Biol Chem, 267; 9: 5968-5976 (1992).
Reynolds, E. C. Journal of Dental Research, 76; 9: 1587-1595 (1997)
Salih et al, Connect Tissue Res 38: 225-235 (1998)
Schlesinger et al Int J Pept Protein Res 30: 257-262 (1987).
Schlesinger, D. H., and Hay, D. I., J Biol Chem 252: 1689-1695 (1977)
Schupbach et al, P., Oppenheim, F. G., Lendenmann, U., Lamkin, M. S., Yao, Y., & Guggenheim, B. European Journal of Oral Sciences, 109; 1: 60-68 (2001).
Schwartz et al, Calcif Tiss Int 50, 511-517 (1992).
Welton, H., in “The functions of salivary proteins” Edgar W M, O'Mullane Dm, Saliva and oral health, Br Dent J 2^{nd} ed., 105-122, (1996)
Yamakoshi et al Arch Oral Biol 46: 1005-14 (2001)
Yao et al Arch Oral Biol 46; 4: 293-303 (2001).

1-21. (canceled)

22. A peptide fragment of statherin comprising formula (I)
X₁X₂X₃X₄X₅X₆X₇X₈X₉X₁₀X₁₁X₁₂X₁₃X₁₄X₁₅X₁₆X₁₇X₁₈X₁₉X₂₀X₂₁ (I)
in which:
each X₁, X₂, X₃, X₄, and X₅ is, independently, an acidic amino acid selected from serine, aspartic acid, and glutamic acid;
X₆ is a basic amino acid selected from lysine, arginine, and histidine;
each X₇ and X₈ is, independently, a hydrophobic amino acid selected from glycine, alanine, phenylalanine, valine, leucine, isoleucine, tyrosine, tryptophan, and methionine;
each X₉ and X₁₀ is, independently, a basic amino acid selected from lysine, arginine, and histidine;
each X₁₁ and X₁₂ is, independently, a hydrophobic amino acid selected from glycine, alanine, phenylalanine, valine, leucine, isoleucine, tyrosine, tryptophan, and methionine;
X₁₃ is a basic amino acid selected from lysine, arginine, and histidine;
each X₁₄ and X₁₅ is, independently, a hydrophobic amino acid selected from glycine, alanine, phenylalanine, valine, leucine, isoleucine, tyrosine, tryptophan, and methionine;
X₁₆ is a tyrosine;
X₁₇ is a hydrophobic amino acid selected from glycine, alanine, phenylalanine, valine, leucine, isoleucine, tyrosine, tryptophan, and methionine;
X₁₈ is a tyrosine;
each X₁₉ and X₂₀ is, independently, a hydrophobic amino acid selected from glycine, alanine, phenylalanine, valine, leucine, isoleucine, tyrosine, tryptophan, proline, and methionine; and
X₂₁ is a hydrophobic amino acid selected from glycine, alanine, phenylalanine, valine, leucine, isoleucine, tyrosine, tryptophan, and methionine.

23. A peptide according to claim 22 wherein the peptide comprises the sequence:
X₁X₂X₃X₄X₅(R/K)₆X₇X₈(R/K)₉(R/K)₁₀X₁₁X₁₂(R/K)₁₃X₁₄X₁₅(Y)₁₆X₁₇(Y)₁₈X₁₉X₂₀(Y)₂₁
wherein the amino acids at positions 6, 9, 10, and 13 are, independently, arginine or lysine, and
wherein the amino acids at positions 16, 18, and 21 are tyrosine.

24. A peptide according to claim 22 wherein:
each X₇, X₈, and X₁₁ is, independently, phenylalanine, leucine, or isoleucine;
X₁₂, X₁₅, X₁₇, and X₁₉ are glycine;
X₁₄ is phenylalanine;
X₂₀ is proline; and
X₂₁ is tyrosine.

25. A peptide according to claim 22 wherein the peptide is selected from:

| | |
|---|---|
| DSSEEKFLRRIGRFGYGYGPY; | (SEQ ID NO:8) |
| DDSEEKFLRRIGRFGYGYGPY; | (SEQ ID NO:9) |
| DDDEEKFLRRIGRFGYGYGPY; | (SEQ ID NO:10) |
| DDDDDKFLRRIGRFGYGYGPY; | (SEQ ID NO:11) |
| DSEEEKFLRRIGRFGYGYGPY; | (SEQ ID NO:12) |
| DEEEEKFLRRIGRFGYGYGPY; | (SEQ ID NO:13) |
| and | |
| EEEEEKFLRRIGRFGYGYGPY. | (SEQ ID NO;14) |

26. A peptide according to claim 22 wherein the peptide is phosphorylated.

27. A peptide according to claim 26 wherein the peptide is selected from:

| | |
|---|---|
| DpSpSEEKFLRRIGRFGYGYGPY; | (SEQ ID NO:8) |
| DDpSEEKFLRRIGRFGYGYGPY; | (SEQ ID NO:9) |
| and | |
| DpSEEEKFLRRIGRFGYGYGPY. | (SEQ ID NO:12) |

28. A peptide according to claim 22 in the form of a fusion protein.

29. A peptide according to claim 22 wherein the peptide consists of formula I.

30. A process of preparing a peptide according to claim 22 comprising ligating successive amino acids together by a solid-state synthetic process or a liquid-phase chemical synthesis reaction, or expression by a nucleic acid construct in a suitable host cell.

31. A pharmaceutical composition comprising a peptide according to claim 22.

32. A pharmaceutical composition according to claim 31 wherein the composition is an artificial saliva, a mouth wash, tooth paste or cream, moisturiser, chewing gum, or drink.

33. A pharmaceutical composition according to claim 31 wherein the composition is an artificial saliva that further comprises an inorganic acid Group I or Group II metal ion salt, and water, and optionally flavoring and/or preservative.

34. A pharmaceutical composition according to claim 31 wherein the composition is an artificial saliva that comprises:
a) 50.0 to 200.0 mg/L of the peptide; 0.5 to 2.0 g/L potassium chloride; 0.5 to 1.5 g/L sodium chloride; 0.05 to 0.20 g/L magnesium chloride; 0.05 to 0.50 g/L calcium chloride; 0.5 to 1.0 g/L potassium hydrogen phosphate; 0.25 to 1.0 g/L potassium dihydrogen phosphate; 1.25 to 2.50 g/L methyl p-hydroxybenzoate; 2.5 to 5.0 g/L flavouring; 35.0 to 50.0 g/L 70% sorbitol; 7.0 to 15.0 g/L sodium carboxymethyl cellulose; and 2.5 to 5.0 mg/L sodium fluoride; or
b) 50.0 to 200.0 mg/L of the peptide; 5 to 15 g/L carboxymethylcellulose; 1 to 10 g/L sorbitol; 1 to 5 g/L potassium chloride; 0.5 to 1.5 g/L sodium chloride; 0.01 to 0.10 g/L magnesium chloride hexahydrate; 0.1 to 0.5 g/L calcium chloride dihydrate; 0.1 to 0.5 g/L potassium hydrogen phosphate; and 0.05 to 0.20 g/L potassium thiocyanate.

35. A pharmaceutical composition according to claim 31 wherein the composition is a mouth wash that further comprises a physiologically acceptable media, a sweetener, a preservative, a flavouring, an anti-bacterial peptide or enzyme, and a detergent.

36. A nucleic acid molecule comprising a nucleotide sequence encoding a peptide of claim 22.

37. A vector comprising the nucleic acid molecule according to claim 36.

38. A cell comprising the vector according to claim 37.

39. A method of preventing or treating demineralisation of teeth, dental disease, or dry mouth syndrome in a subject comprising administering to the subject a peptide according to claim 22.

40. A method according to claim 39 wherein the dental disease is dental caries.

41. A method according to claim 39 wherein the dental disease is dental erosion.
